(19) United States
(12) Patent Application Publication
(10) Pub. No.: US 2026/0157746 A1
(43) Pub. Date: Jun. 11, 2026

(54) Tensionable Anchors and Methods of Tissue Repair

(71) Applicant: Arthrex, Inc., Naples, FL (US)

(72) Inventors: Jason A. VALENTIN, Fort Myers, FL (US); Thomas DOONEY, JR., Naples, FL (US); Matthew R. HERRINGTON, Naples, FL (US)

(73) Assignee: Arthrex, Inc., Naples, FL (US)

(21) Appl. No.: 19/367,002

(22) Filed: Oct. 23, 2025

Related U.S. Application Data
(60) Provisional application No. 63/730,093, filed on Dec. 10, 2024.

(51) Int. Cl. A61B 17/04; A61B 17/06; A61F 2/08

(52) U.S. Cl. CPC A61B 17/0466; A61B 17/06166; A61F 2/0811; A61B 2017/0464; A61B 2017/0618; A61F 2002/0852

(57) ABSTRACT

Surgical constructs and methods for surgical repairs are disclosed. A surgical construct is in the form of a soft suture anchor with flexible coupler terminating in two opposing ends. One of the two ends forms at least one splice and anchoring loops formed with the other end.

### CROSS-REFERENCE TO RELATED APPLICATIONS

This application claims priority to US Provisional Application No 63/730,093 filed on Dec. 10, 2024, the disclosure of which is hereby incorporated by reference in its entirety herein.

### BACKGROUND

The disclosure relates to the field of surgery and, more specifically, to surgical anchor constructs and associated methods of tissue repairs.

### SUMMARY

Surgical systems, assemblies, and methods for fixation of soft tissue are disclosed.

A surgical construct may create a self-locking, reinforced repair. A surgical construct may be made completely of suture to achieve fixation in bone without a separate anchoring body and with increased fixation. The design enables fixation by deployment into bone with a suture tail remaining outside the bone for tensioning and/or alternative usage. The surgical construct may be employed in fixation of first tissue to second tissue, for example, fixation of soft tissue to bone.

Methods of tissue repairs are also disclosed. In some implementations, a surgical construct may provide tissue fixation without any knot formation, by providing an all-suture soft anchor that does not require a separate anchoring body or similar structure, and which allows increased tissue fixation with even and broad tissue compression.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates an exemplary method of assembling a surgical construct at an initial stage.

FIG. 2 illustrates the surgical construct of FIG. 1 at a subsequent stage.

FIG. 3 illustrates the surgical construct of FIG. 2 at a subsequent stage.

FIG. 4 illustrates another view of the surgical construct of FIG. 3.

FIG. 5 illustrates the surgical construct of FIG. 4 undergoing tensioning.

FIG. 6 illustrates the surgical construct of FIG. 5 undergoing tensioning.

FIG. 7 illustrates a top view of the surgical construct of FIG. 3 positioned on an inserter.

FIG. 8 illustrates a side view of the surgical construct of FIG. 3 positioned on an inserter.

FIG. 9 illustrates a repair with surgical constructs.

FIG. 10 illustrates another repair with surgical constructs.

### DETAILED DESCRIPTION

A surgical construct may create a reinforced, self-locking repair.

A tissue repair system may include a self-locking surgical construct with a fixation device in the form of a flexible coupler. A flexible coupler may be suture. A flexible coupler may be a round-flat-round suture construct. A flexible coupler may be suture tape. A suture tape may be a flat-braided SutureTape with ends formed of round suture. The SutureTape may be a FiberTape® suture. The SutureTape may be a TigerTape® suture. A tissue repair system may be a soft tissue repair system.

In some implementations, a soft suture anchor may include a flat-braided suture tape provided with round suture on each end of the suture tape. One end of the suture tape may be dedicated to (and act as) the anchoring body. The other end can be modified to complement auxiliary instrumentation or implants.

Methods of tissue repairs are also disclosed. In some implementations, a surgical construct may provide first tissue to second tissue fixation, for example, fixation of soft tissue (ligament, tendon, graft, etc.) to bone in a simple and fast manner.

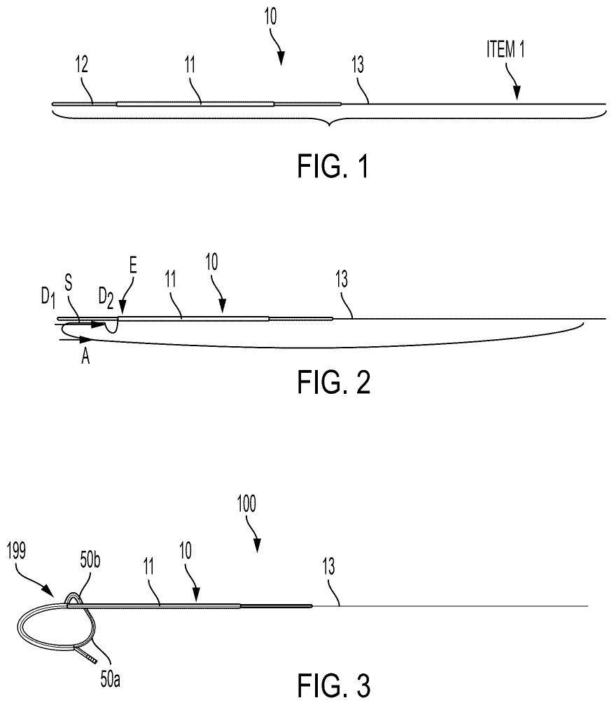

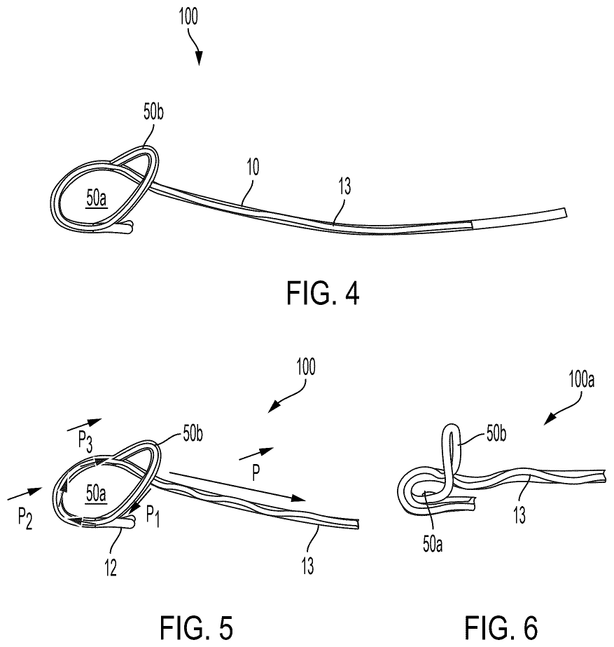

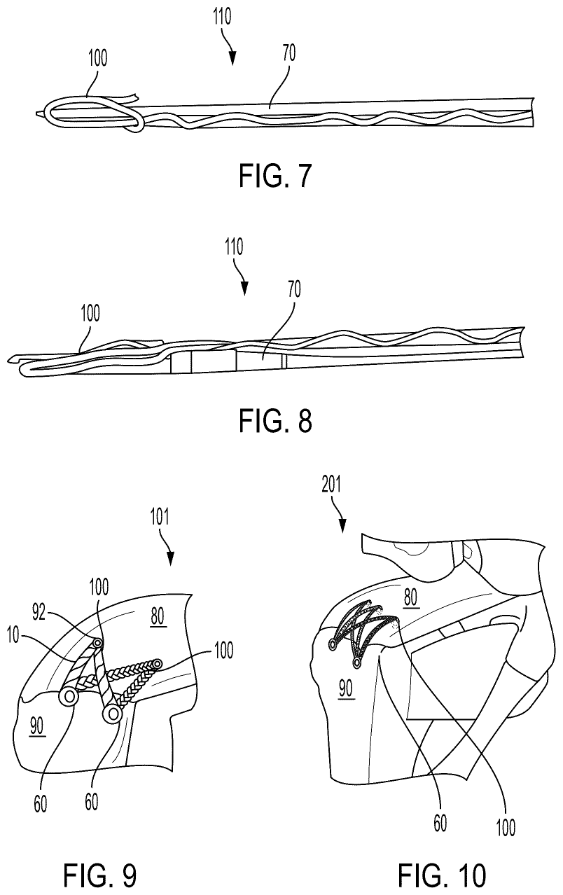

Referring now to the drawings, where like elements are designated by like reference numerals, FIGS. 1-3 illustrate exemplary steps of assembling surgical construct 100 including fixation device 10 (suture, braid or suture tape 10) with exemplary anchoring mechanism 199. FIGS. 4-6 illustrate the bunching effect of the surgical construct 100. FIGS. 7 and 8 illustrate surgical construct 100 positioned on an inserter instrument. FIGS. 9 and 10 illustrate exemplary soft tissue repairs 101, 201 with surgical construct 100.

Surgical construct 100 may be an implantable device; a surgical assembly; a surgical system; a suture tape anchor; an anchoring construct; an anchoring, self-locking, surgical anchor; a single limb soft anchor; a single limb FiberTak® construct; or a SutureTape FiberTak® fixation device. Implant 100 may be supported by a small bone hole and may produce a single fixed repair limb for a custom tissue to bone repair. In some implementations, the device may consist of a single suture with a lumen that is configured in such a way as to create an anchoring body that bulks or bunches up when inserted into a bone hole. The single repair limb may be used for additional procedures, for example, may be knotted with additional flexible couplers and/or may be employed to be secured within different tissue locations with fixation devices such as knotted or anchoring suture anchors.

As detailed below, surgical construct 100 may be an implantable device made completely of suture or sutures that achieves fixation in bone without a separate anchoring body. The design enables for fixation by deployment into bone with a suture tail remaining outside the bone for tensioning and alternative usage.

Surgical construct 100 of FIG. 3 includes fixation device 10 in the form of suture tape 11 provided with two ends 12, 13. Suture tape 11 may be flat-braided SutureTape with ends 12, 13 formed of round suture.

Fixation device 10 may be a suture tape; a braid; a suture; a repair suture; a round-flat suture; or a round-flat-round suture. In some implementations, suture tape 11 may have a length of about 20 to about 40 inches (for example, of about 36 inches), whereas each of ends 12, 13 may have a length of about 1 to about 6 inches (for example, of about 3 inches).

In some implementations, fixation device 10 may be provided as a one-piece machine taper construct in the form of a round to flat to round construct. The fixation device 10 acts as a soft anchor. In some implementations, fixation device 10 may be a simple round suture with ends formed also of round suture, of similar or different diameters. In some implementations, fixation device 10 may be a round suture with two tapered ends. In some implementations, fixation device 10 may be a round suture with a single tapered end. In some implementations, fixation device 10 may be a simple round suture. FIG. 1 illustrates an exemplary-only embodiment wherein fixation device 10 includes a suture tape 11 terminating with tapered end 13 (having at least two different regions with different diameters) and end 12 (which may also be a tapered end). Ends 12, 13 may be tapered ends formed by splicing bifurcated, trifurcated, quadfurcated etc. ends through the unspliced ends and cutting the remaining ends.

Although, for simplicity, the implementations below will be described with reference to device 10 in the form of a round-flat-round suture, it must be understood that the disclosure is not limited to these exemplary-only implementations and encompasses any type of fixation device 10.

The fixation device 10 may be manufactured from any flexible material, for example, multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). Surgical constructs may be used with any type of flexible material or suture known in the art. The fixation device 10 may include elastic material. The fixation device 10 may consist of elastic tapered suture.

Reference is now made to FIG. 2. The tip of at least one of ends 12, 13, preferably the tip of each of ends 12, 13, may be glued. In some implementations, at least one of glued tips may have a length of about 0.5 to about 1 inch.

To form loops 50a, 50b, an exemplary shuttling device such as a needle or Nitinol loop (not shown) may be passed within round end 12 from a first location D1 to a second location D2 in the direction of arrow A (FIG. 2). To allow formation of a splice of end 13 within end 12, the needle enters end 12 at first location D1, advances along a longitudinal axis of end 12 and in the direction of arrow A and exits the end 12 at second location D2.

Once the needle has exited end 12 at second location D2, the needle then pierces again and exits tapered end 12 at pierce/exit point E (third location E) (FIG. 2) located along the longitudinal axis of end 12 and adjacent the suture tape 11. Exit location E may be about coincidental with most left end of suture tape 11. Thin tapered end 13 of flexible coupler 10 may then be attached to the shuttle/pull device (for example, threaded onto the needle) and passed and spliced through the left end 12 at splice S in the left end 12 (FIG. 3) and exiting the left end at exit point E. Tapered end 13 may be spliced into opposite end 12 for about 10 mm to about 30 mm, preferably for about 20 mm (e.g., length of splice S). End 13 may exit the splice on the same side as the splice entered and then may pierce again through end 12. In some implementations, end 13 may pierce again through the end 12 after about 7 to 9 mm from end of splice S, preferably about 8 mm after.

When the needle is pulled out of the fixation device 10, tapered end 13 forms two loops with end 12: a first loop 50a and a second loop 50b. Loop 50a may be a flexible, closed, continuous loop. Loop 50b may be a flexible, closed, continuous loop. Loop 50a is adjacent loop 50b, and as part of anchoring mechanism 199 (FIG. 3). Once inserted into bone, and as detailed below, loop 50a may be reduced (e.g., the perimeter and length of loop 50a may be reduced) by pulling on the exposed suture limb 13 to allow loop 50a to collapse.

FIG. 3 illustrates surgical construct 100 in the assembled form and as provided to the surgeon (pre-assembled and ready for insertion). Surgical construct 100 may be inserted into bone via a self-punching driver or by any other means to allow insertion into a drilled hole in bone 90, and as detailed below.

FIGS. 4-6 illustrate tensioning of construct 100 while inserted into a bone hole. Pulling on the exposed suture, allows loop 50a to reduce its length and perimeter and to collapse within the bone hole. FIG. 4 illustrates surgical construct 100 in unbunched or relaxed, untensioned state. FIGS. 5 and 6 illustrate bunching of construct 100 of FIG. 4 by pulling free end 13 in the direction of arrow P and traveling in the sense/direction of arrows P1, P2, P3 to reduce the diameter and length of loop 50a and to form, therefore, the bunched shape of bunched, tensioned construct 100a shown in FIG. 6.

FIGS. 7 and 8 illustrate a top view and a side view of surgical construct 100 secured to inserter 70 and forming surgical assembly 110.

First and second loops 50a, 50b may be closed, flexible, continuous loops that are adjacent and may be in contact with each other, and that also extend along a longitudinal axis of the flat suture tape 11 and end 12 to form a construct similar to that of a figure “8” (e.g., are located along the longitudinal axis of tape 11 in a FIG. 8 configuration). First and second loops 50a, 50b may have similar or different lengths and/or radii and may have an exemplary circular or semi-circular configuration. Loop 50a may be a closed, adjustable, continuous loop having an adjustable perimeter. As detailed above, loop 50a bunches up when end 13 is pulled to decrease the length and perimeter of loop 50a and to bunch up the surgical construct 100. Once tensioning of the construct has occurred, free end 13 may be cut. Alternatively, suture end 13 may be used for other tissue repairs that may include knotted or anchoring repairs, such as lateral tissue compression with accompanying interference anchors, for example. By providing suture tape 11 and end 13 over soft tissue, construct 100 provides additional tissue fixation allowing the tissue to lay flat and evenly throughout the repair and across the bone, for example. In this manner, one or more constructs 100 may provide flat and broad soft tissue compression, particularly for shoulder repairs.

Although the implementations above have been described with reference to particular embodiments wherein only one loop 50a and one loop 50b have been formed, it must be understood that the disclosure is not limited to these exemplary-only implementations. Thus, the disclosure also contemplates surgical constructs having three or more loops. In these implementations, the shuttle/pull device (needle, for example) may pass through the end 12 at various regions (splices) and may exit the end 12 at multiple exit points/locations to form a plurality of loops 50a, 50b. A plurality of loops 50a, 50b provides increased fixation into bone as well as increased repair strength.

In additional implementations, end 13 may be a furcated end with multiple limbs (for example, a trifurcated end with three exemplary limbs having similar or different diameters and/or configurations). Each limb may be spliced through end 12 as detailed above and each end may form one loop 50a and one loop 50b. Once the device has been introduced and secured into bone, each of the multiple limbs may be together or separately passed over tissue and be secured thereof, to allow for even tissue compression onto bone and increased fixation.

Reference is now made to FIGS. 9 and 10 which illustrate schematic tissue repairs 101, 201 (e.g., tendon or ligament repair) with exemplary surgical construct 100, to secure a first tissue 80 (for example, soft tissue such as tendon 80) to a second tissue 90 (for example, bone 90).

Once surgical construct 100 has been inserted and secured within a hole 92 in bone 90, the flexible, free end 13 may be pulled to shrink the construct and reduce loop 50a. Suture tape 11 and end 13 may then be passed over soft tissue 80 to be secured to bone 90 to compress the tendon to bone. As noted, each surgical construct may include one or more repair limbs to allow for even and increased tissue compression.

Suture tape 11 and end 13 may then be knotted with additional flexible couplers or may be secured to bone 90 with additional fixation devices 60, providing a final repair 101, 201 with increased compression of tissue. FIG. 9 illustrates repair 101 with two surgical constructs 100. FIG. 10 illustrates repair 201 with three surgical constructs 100. Any number of surgical constructs 100 may be employed with any number of repair limbs 13, depending on the characteristics of the repair and surgeon's preferences.

Fixation device 60 may be any implant, button, anchor, for example, knotted anchor, knotless anchor, or all-suture anchor, or any device that confers secure attachment and fixation of soft tissue 80 over bone 90. The fixation device may be an anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. The fixation device may be an all-suture soft anchor. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

The constructs, systems, and assemblies of the present disclosure may be employed in numerous soft tissue repairs and fixations, for example, fixation of soft tissue to bone.

A surgical construct 100 may comprise a fixation device 10 in the form of a round-flat-round suture tape construct 10; at least one loop 50a and at least one loop 50b formed with and within the fixation device 10. The surgical construct 100 may be a soft anchor. The surgical construct 100 may be a unitary construct. The at least one loop 50a may be a knotless, closed, continuous loop. The flexible coupler 10 may be a flat suture tape 11 or flat braid 11 terminating in round suture 12, 13. One of ends 12, 13 may form at least one splice S within the flexible coupler 10. One of ends 12, 13 may be tensioned to bunch up the surgical construct 100 within the bone hole (and to pull out any slack in the bone hole) and may then be employed for additional surgical steps such as soft tissue compression. One of ends 12, 13 may be passed over soft tissue 80 and may be secured at a different location in bone 90, separate from bone hole 92. One of ends 12, 13 may be secured into bone 90 with a fixation device 60.

Surgical construct 100 may be positioned on an inserter instrument 70 as part of surgical assembly 110.

A soft anchor 100 may include a flat suture tape 11 terminating into two ends 12, 13. At least one of the two ends 12, 13 may be round suture. One of two ends 12, 13 may form at least one splice S within the other end and the suture tape 11; at least one loop 50a; and at least one loop 50b adjacent loop 50a, and as part of anchoring mechanism 199. The at least one loop 50a may be a knotless, closed, flexible, continuous loop with an adjustable perimeter. The at least one loop 50b may be a knotless, closed, flexible, continuous loop with a fixed perimeter and length. One of two ends 12, 13 may form a knot with additional flexible coupler(s). One of two ends 12, 13 may be passed over soft tissue and secured within hard tissue. One of two ends 12, 13 may be secured to hard tissue with a knotted or anchoring fixation device.

Methods of soft tissue repair 101, 201 which provide increased, even, flat soft tissue compression with respect to the bone, are also disclosed.

A method of tissue repair 101 may comprise inter alia the steps of: (i) securing a surgical construct 100 into a first tissue 90, the surgical construct being preformed with a suture tape 11; at least a first loop 50a; at least a second loop 50b adjacent the at least the first loop 50a; and a free end 13; and (ii) passing the free end 13 over a second tissue 80 to be positioned relative to the first tissue 90, to compress the second tissue 80 over the first tissue 90. The method may further include (iii) securing the free end 13 to the first tissue 90 with a fixation device 60. The fixation device may be a knotted anchor. The tissue repair may be a knotted repair.

As detailed above, when the suture tape 11 is inserted into bone 90, the suture tape resides within the bone and acts as a soft anchor (as a body of a soft anchor). The repair suture limb(s) resides on top of the bone. The repair suture is passed over the tissue, and then secured to hard tissue with additional fixation devices such as fixation device 60. The steps may be repeated for each additional surgical construct provided at the repair site and/or for each repair limb of each surgical construct.

Suture tape 11 and ends 12, 13 may include any flexible material, for example, multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM) fibers, braided with at least one other fiber, natural or synthetic, to form lengths of suture material.

Suture tape 11 and ends 12, 13 may be also formed of a stiff material, or combination of stiff and flexible materials, particularly for the regions of the coupler that are passed/spliced through the body of the coupler and depending on whether they are employed with additional fixation devices. Suture tape 11 and ends 12, 13 may be also coated and/or provided in different colors for easy manipulation during the surgical procedure. The constructs of the present disclosure may be used with any type of flexible material or suture that may be weaved or passed through itself.

Various structural elements of surgical construct 100 may be visually coded, making identification and handling of the sheath and suture legs simpler. Easy identification of suture in situ is advantageous in surgical procedures, particularly during arthroscopic surgeries, endoscopic and laparoscopic procedures.

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A soft anchor, comprising:
a flexible coupler with a first end and an opposite second end; and
a first loop adjacent a second loop, wherein the first loop and the second loop are formed by passing one of the first and second ends through the other of the first and second ends.

2. The soft anchor of claim 1, wherein the flexible coupler is a suture tape and wherein each of the first and second ends is round suture.

3. The soft anchor of claim 2, wherein the suture tape and the first and second round sutures are formed as a one-piece construct.

4. The soft anchor of claim 1, wherein the one of the first and second ends forms at least one splice when passed through the other of the one of the first and second ends.

5. The soft anchor of claim 1, wherein the first and second loops are knotless, closed, continuous loops.

6. The soft anchor of claim 1, wherein the first loop and the second loop are formed by piercing one of the first and second ends with the other of the first and second ends for a plurality of times and at a plurality of piercing points along a length of the one of the first and second ends.

7. The soft anchor of claim 1, wherein the flexible coupler consists essentially of elastic suture.

8. A soft anchor, comprising:
a suture tape; and
a first round suture at one end of the suture tape, and a second round suture at another end of the suture tape,
wherein the first round suture forms at least one splice and at least two loops with the second round suture.

9. The soft anchor of claim 8, wherein the at least two loops are adjacent each other.

10. The soft anchor of claim 8, wherein the suture tape and the first and second round sutures are formed as a one-piece machine taper construct.

11. The soft anchor of claim 8, wherein the suture tape consists essentially of elastic suture.

12. The soft anchor of claim 8, wherein the first round suture has a diameter similar to a diameter of the second round suture.

13. The soft anchor of claim 8, wherein the first round suture has a diameter different from a diameter of the second round suture.

14. The soft anchor of claim 8, wherein at least one of the first round suture and the second round suture is tapered.

15. The soft anchor of claim 8, wherein the suture tape forms the at least two loops.

16. The soft anchor of claim 8, wherein the at least two loops are configured to bunch up when inserted into bone.

17. The soft anchor of claim 8, wherein the soft anchor consists of the suture tape; the first and second round sutures; and the at least two loops.

18. A method of tissue repair comprising:
securing, in a first tissue, a surgical construct consisting of a flat suture tape terminating into a first round suture at one end and a second round suture at another end, wherein one of the first and second round sutures forms at least one splice and at least two loops with the other of the first and second round sutures; and
passing the other of the first and second round sutures over a second tissue to be positioned relative to the first tissue to compress the second tissue over the first tissue.

19. The method of claim 18, wherein the first tissue is bone and the second tissue is tendon or ligament.

20. The method of claim 18, wherein the surgical construct is an all-suture anchor formed of a single suture material.

21. The method of claim 18, further comprising securing the other of the first and second round sutures into the first tissue with a fixation device, and after compressing the second tissue over the first tissue.

22. The method of claim 21, wherein the fixation device is a knotted anchor.

23. The method of claim 18, wherein the at least two loops are closed, continuous, knotless loops adjacent to each other.

24. A method of assembling a soft anchor, comprising:
passing a first end of a flexible coupler through a second end of the flexible coupler, to form at least one splice and two anchoring, closed, continuous loops within the second end,
wherein one of the two anchoring, closed, continuous loops is adjacent another of the two anchoring, closed, continuous loops.

25. The method of claim 24, wherein the flexible coupler is a suture tape and wherein each of the first and second ends is round suture.

26. The method of claim 24, wherein the two anchoring, closed, continuous loops are configured to be inserted into bone and to act as a body of the soft anchor.
